(19) United States
(12) Patent Application Publication
(10) Pub. No.: US 2003/0236530 A1
(43) Pub. Date: Dec. 25, 2003

(54) Adjustable fluid flow resistor

(76) Inventors: Laurence J. Cull, Wildwood, MO (US); William J. Neubert, Ballwin, MO (US)

Correspondence Address: Michael L. Smith, Bausch & Lomb Inc., One Bausch & Lomb Place, Rochester, NY, 14604-2701, US

(21) Appl. No.: 10/179,660

(22) Filed: Jun. 24, 2002

(51) Int. Cl. A61F 9/00

(52) U.S. Cl. 606/107

(57) ABSTRACT

An adjustable fluid flow resistor 10 for use in ophthalmic surgery includes a housing 12, a first set of dividers 24, and a second set of dividers 28, contained without the housing 12. Fluid flow resistance is adjustable between a minimum when the apertures 26 and 30 of the first and second set of dividers 24 and 28 are aligned. The flow resistance increases as the apertures 26 of the first set of dividers 24 are positioned further away from the apertures 30 of the second set of dividers 28.

### BACKGROUND OF THE INVENTION

1. Field of the Invention

The present invention is related to devices for restricting the flow of aspirant during surgery, especially ophthalmic surgery. In particular, the present invention relates to devices that are adjustable so as to provide a range of aspirant flow resistance.

2. Description of Related Art

During eye surgery, especially cataract surgery, surgeons experience a tension between the amount of vacuum or aspiration to be used on a patient's eye and the time period in which the surgeon has to respond to events that may occur during surgery. Surgeons typically prefer higher vacuum levels to provide a higher holding force for the cataract. However, these higher vacuum levels result in the need for rapid response times by the surgeon when events such as occlusion occur in the aspiration line. The higher the vacuum levels, the quicker events occur and thus the potential for serious problems increases, such as the tearing of the capsular bag.

There are known devices for increasing the resistance to aspirant fluid flow to allow a surgeon to use higher vacuum levels, i.e., higher holding force, with a slower response time. These devices help the surgeon have the benefits of higher vacuum levels while limiting or minimizing the risks by providing the surgeon with greater time to respond to surgical events than would be possible without resistance to the aspirant flow. Coiled tubing is one example that increases the flow resistance. It has been asserted that increased resistance is achieved by passing fluid through a series of coil bends because fluid drops in pressure as it flows through a bend. However, a downside to the coiled tubing is that the chances of aspirant clogging within the coils is increased due to the elliptical cross section and bent kinks that may occur in the tubing. In addition, the resistance of the coiled tubing is a function of the coil radius and the resistance cannot be varied during surgery.

Another device that increases resistance to aspirant flow is a non-clogging orifice that collects waste and is commonly referred to phaco-guard. The phaco-guard is a large cross-sectional area filter funneled down to a small orifice. It allows limited clogging of the filter and is based on the assumption that the entire filter area will not clog. The filter may still clog and it is not adjustable.

Therefore, it would be advantageous to have a fluid flow resistor that is adjustable to provide the surgeon with a range of aspirant flow resistance.

### BRIEF DESCRIPTION OF THE DRAWINGS

FIG. 1 is a cross-section of an adjustable fluid flow resistor in accordance with the present invention;

FIG. 2 is a front elevation view of an adjustable fluid flow resistor in accordance with the present invention wherein the fluid flow resistance is at a minimum;

FIG. 3 is a front elevation view of an adjustable fluid flow resistor in accordance with the present invention wherein the resistor is in a position of maximum resistance to fluid flow;

FIG. 4 is an illustration of the use of the present invention during eye surgery.

### DETAILED DESCRIPTION OF THE PREFERRED EMBODIMENT

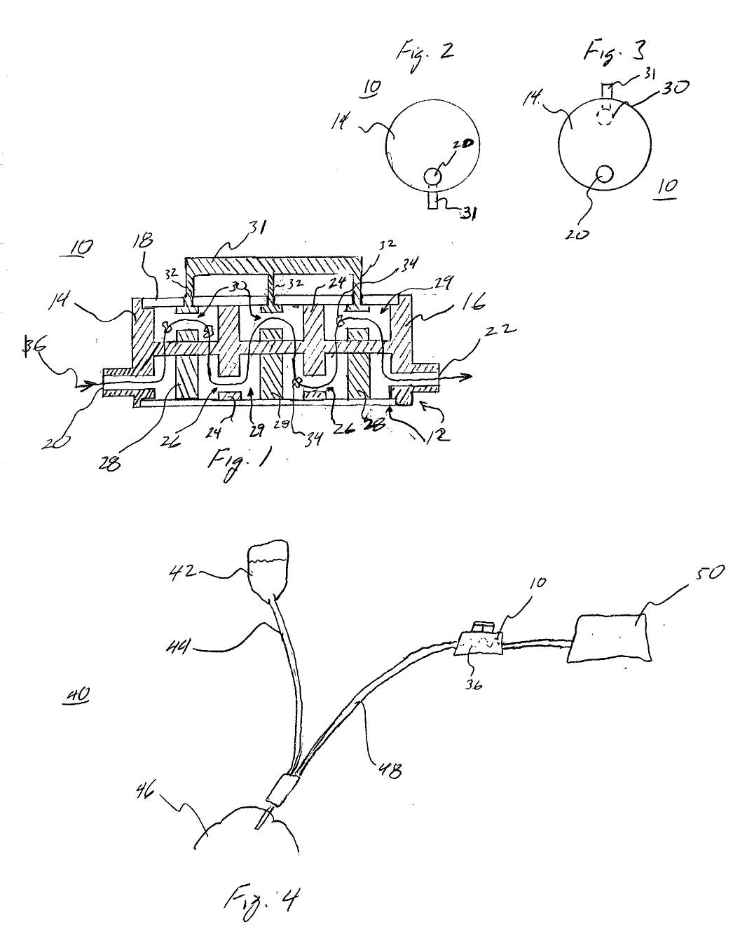

FIG. 1 shows an adjustable fluid flow resistor 10 in accordance with the present invention. The resistor 10, which is for use in ophthalmic surgery, includes a housing 12 formed of inlet 14 and outlet 16 attached to opposing ends of tube 18. Inlet 14 and outlet 16 are to be connected to surgical tubing for receiving aspirant from a surgical site and allowing the aspirant to pass to a container as described below. The material used to form inlet 14, outlet 16, and tube 18 may be any material suitable for ophthalmic surgery. Inlet orifice 20 and outlet orifice 22 are both preferably large enough in cross-section so that any aspirant flowing through attached surgical tubing will not clog in the orifices 20 and 22. It is also to be understood that the resistor 10 could be made integral to a surgical fluid collection cassette such as are well-known in the art. If the resistor is incorporated into a cassette it may also be desirable to automate the adjustment of the flow resistance.

Contained within housing 12 is a first set of dividers 24. Each of the first set of dividers 24 includes at least one aperture 26 defining a path through which aspirant may flow during surgery. Dividers 24 should preferably be sealingly attached tube 18 so that fluid and aspirant may only flow through apertures 26.

A second set of dividers 28 is also contained within the housing 12. The dividers 28 are moveable relative to the first set of dividers 24 and each of the second set of dividers 28 includes at least one aperture 30 defining a path through which aspirant may flow during surgery. Dividers 28 should also be sealingly engaged with tube 18, but unlike stationary dividers 24, dividers 28 are preferably rotatable within resistor 10 by manipulation of handle 31. Of course, dividers 24 may also be made to be rotatable within resistor 10. The dividers 24 and 28 within housing 12 are spaced apart from each other to define a plurality of aspirant chambers 29.

A flow resistance of resistor 10 is at a minimum when the apertures 26 and 30 of the first and second set of dividers 24 and 28 are aligned and the flow resistance increases as the apertures 26 and 30 of the first set of dividers 24 are positioned further away from the second set of dividers 28. The fluid flow resistor 10 of FIG. 1 is shown at a maximum resistance position, where the apertures 30 are 180° from apertures 26.

Handle 31 includes arms 32 which are connected to dividers 28. Obviously, arms 32 passing through tube 18 must be formed such that a liquid-tight seal is made between arms 32, divider 28, and tube 18 so that no surgical fluid escapes from resistor 10 during surgery.

It is preferred that the orifices 20 and 22 and the apertures 26 and 30 and the distance between the dividers 28 and 24 are all large enough such that pieces of aspirant 34 do not become clogged within resistor 10 during surgery, and are able to flow freely through resistor 10. Said another way, each apertures 26 and 30 and the distance between each of the first and second set of dividers 24 and 28 is at least as large in cross-section as a cross-section of an inner-diameter of tubing to be connected to the inlet, thereby avoiding any possibility of aspirant occluding one of the apertures 26 and 30 or between any dividers. It is preferable for the spacing to cause turbulent flow, which is known to have higher resistance than laminar flow.

While the preferred embodiment shows only one aperture per divider, it is to be understood that more than one aperture could be contained in the dividers 24 and 28. The apertures could also be notches formed in the dividers 24 and 28 or a variety of other spacings.

The handle 31 connected to the second set of dividers 28 allows a user to rotate the second set of dividers 28 to vary the fluid flow resistance. However, other ways of rotating the dividers can also be used. For example, a motor could be utilized for rotation or each divider could have its own independent handle. Also, other means of movement of the dividers may be used such as hinging each of the dividers or any other manner of varying the fluid flow path length. Such hinging movement is described in co-pending application entitled “Adjustable Fluid Flow Resistor Cassette, serial number (to be assigned) and is incorporated herein by reference.

FIG. 2 shows the aspirant flow resistor 10 positioned such that the resistance of resistor 10 is minimal. FIG. 3 shows the resistor 10 positioned at its maximum resistance where apertures 30 are rotated 180° from inlet 20 and apertures 26. This causes aspirant to flow along line 36 as shown in FIG. 1 which creates an increased fluid flow path length from the configuration shown in FIG. 2.

Another way of describing the invention follows. Housing 12 has an inlet 20 and an outlet 22 for receiving a flow of aspirant fluid for a surgical site. The first and second set of dividers 24 and 28 together form a set of path restrictors, and are disposed within the housing between the inlet 20 and outlet 22. At least some of the path restrictors are moveable within the housing, and cooperate to vary a path length 36 from the inlet to the outlet. An aspirant fluid flow resistance increases as the path length 36 increases from the inlet to the outlet.

FIG. 4 shows the present invention is use in ophthalmic surgery. Eye surgery 40 includes irrigation fluid 42 passing to the surgical site through tube 44 and into eye 46. Aspirant, including for example pieces of removed cataract, and irrigation fluid 42 pass through tube 48 and into aspirant flow resistor 10 and then onto a collection reservoir 50. The present invention allows a surgeon a great amount of control over the level of vacuum that may be used as well as the response time that will be available to respond to surgical events. Resistor 10 is easily adjusted during surgery by simply rotating handle 31 about the housing 12.

Thus, there has been shown an adjustable aspirant flow resistor in accordance with the claims of the present invention. It is to be understood that changes and alterations may be made to the present invention without departing from the scope of the invention as set forth in claims. For example, dividers 28 may be rotated within the resistor 10 by use of a knob or wheel contained at one end of resistor 10 instead of the disclosed handle 31.

We claim:

1. An adjustable fluid flow resistor for use in ophthalmic surgery comprising:
a housing having an inlet and an outlet for receiving a flow of aspirant fluid from a surgical suite wherein the fluid flows from the inlet to the outlet;
a set of path restrictors disposed within the housing and are moveable within the housing and between the inlet and outlet such that at least some of the path restrictors cooperate to vary a path length from the inlet to the outlet; and
wherein an aspirant fluid flow resistance increases as the path length increases from the inlet to the outlet.

2. An adjustable fluid flow resistor for use in ophthalmic surgery comprising:
a housing having an inlet and an outlet such that the inlet and outlet are to be connected to surgical tubing for receiving aspirant from a surgical site and allowing the aspirant to pass to a container;
a first set of dividers contained within the housing such that each of the first set of dividers includes at least one aperture defining a path through which aspirant may flow during surgery;
a second set of dividers contained within the housing and movable relative to the first set of dividers such that each of the second set of dividers includes at least one aperture defining a path through which aspirant may flow during surgery; and
wherein a flow resistance is at a minimum when the apertures of the first and second sets of dividers are aligned and where the flow resistance increases as the apertures of the first set of dividers are positioned further away from the apertures of the second set of dividers.

3. The resistor of claim 2 further including a handle connected to the second set of dividers for allowing a user to rotate the second set of dividers to vary the fluid flow resistance.

4. The resistor of claim 2 wherein each aperture of each of the first and second set of dividers is at least as large in cross-section as a cross-section of an inner-diameter of tubing to be connected to the inlet, thereby avoiding any possibility of aspirant occluding one of the apertures.

5. The resistor of claim 2 wherein each of the dividers is spaced apart from each other by a distance at least equal to a distance of a cross-section of an inner-diameter of tubing to be connected to the inlet, thereby avoiding any possibility of aspirant becoming clogged between any dividers.

6. The resistor of claim 2 wherein each set of dividers includes at least two (2) dividers.

7. The resistor of claim 2 wherein each divider includes more than one (1) aperture.

8. A variable aspirant flow resistor for use in ophthalmic surgery comprising:
a housing having an inlet and an outlet such that aspirant from a surgical site passes from the inlet and through the outlet;
first and second sets of dividers contained with the housing and spaced apart from each other such that the dividers and the housing define a plurality of aspirant chambers within the housing;
wherein every other divider from the inlet to the outlet belongs to one of the first or second sets of dividers;
wherein each divider includes at least one aperture such that the apertures of each divider taken together define an aspirant flow path from the inlet to the outlet; and
wherein the first and second sets of dividers are rotatable with respect to each other for varying a resistance to aspirant flow such that when each of the apertures of the first set of dividers are aligned with each of the apertures of the second set of dividers a minimum resistance to aspirant flow is achieved and the further each of the apertures of the first set of dividers are rotated away from each of the apertures of the second set of dividers the resistance to aspirant flow increases.

9. The resistor of claim 8 further including a handle connected to the second set of dividers allowing a user to rotate the second set of dividers to vary the fluid flow resistance.

10. The resistor of claim 8 wherein each aperture of each of the first and second set of dividers is at least as large in cross-section as a cross-section of an inner-diameter of tubing to be connected to the inlet, thereby voiding any possibility of aspirant occluding one of the apertures.

11. The resistor of claim 8 wherein each of the dividers is spaced apart from each other by a distance at least equal to a distance of a cross-section of an inner-diameter of tubing to be connected to the inlet, thereby avoiding any possibility of aspirant becoming clogged between any two dividers.
